(19) United States
(12) Patent Application Publication
(10) Pub. No.: US 2022/0022787 A1
(43) Pub. Date: Jan. 27, 2022

(54) INSERTING DEVICE AND BLOOD COLLECTION DEVICE

(71) Applicant: Wen-Bin Shen, Taipei City (TW)

(72) Inventor: Wen-Bin Shen, Taipei City (TW)

(73) Assignee: COMPAL ELECTRONICS, INC., Taipei City (TW)

(21) Appl. No.: 17/382,355

(22) Filed: Jul. 22, 2021

Related U.S. Application Data
(60) Provisional application No. 63/055,338, filed on Jul. 23, 2020.

(51) Int. Cl. A61B 5/151; A61B 5/15

(52) U.S. Cl. CPC A61B 5/15126; A61B 5/150267

(57) ABSTRACT

An inserting device includes a main casing, a bracket, a first elastic component, an inserting assembly and a second elastic component. The main casing has a position-limiting portion and a first holding portion. The bracket is disposed in the main casing and has a second holding portion. The first elastic component is disposed between the main casing and the bracket, and the first holding portion holds the bracket. The inserting assembly is disposed in the main casing. The second elastic component is disposed between the inserting assembly and the bracket. The position-limiting portion limits the second holding portion, such that the second holding portion holds the inserting assembly on the bracket. When the bracket is released by the first holding portion, the bracket, the second elastic component and the inserting assembly move by the elastic force of the first elastic component, the second holding portion is departed from the position-limiting portion, and the inserting assembly is restored by an elastic force of the second elastic component. Moreover, a blood collection device including the inserting device is also provided.

### CROSS-REFERENCE TO RELATED APPLICATION

This application claims the priority benefit of U.S. provisional application Ser. No. 63/055,338, filed on Jul. 23, 2020. The entirety of the above-mentioned patent application is hereby incorporated by reference herein and made a part of this specification.

### BACKGROUND

### Technology Field

The disclosure relates to an inserting device and a liquid collection device with the same, and more particularly to an inserting device and a blood collection device with the same.

### Description of Related Art

Diabetes is a clinical syndrome caused by factors such as absolute or relative lack of insulin in the body, abnormal secretion time, and disorder or resistance of the insulin acting body. If diabetes is not well controlled, it may cause some acute complications, such as hypoglycemia, ketoacidosis, and non-ketone hyperosmolar coma. Serious long-term complications include cardiovascular disease, chronic renal failure, retinopathy, neuropathy, and microvascular disease. For diabetic patients, it is very important to monitor blood sugar from time to time. The primary goal of managing diabetes is to maintain a normal blood sugar level. If the patient can pay attention to blood sugar control, it may be able to effectively prevent the complications.

Before measuring the blood glucose concentration value, a blood collection step must be performed. Blood collection is an invasive sampling method, the process is complicated, and the user feels pain each time when the blood is collected. Therefore, it is necessary to improve the design of the blood collection device to facilitate the use and increase the willingness of users to collect blood by themselves.

### SUMMARY

The disclosure provides an inserting device and a blood collection device with the same, which has better convenience in use and is capable of increasing the willingness of users to collect blood by themselves.

The inserting device of the disclosure includes a main casing, a bracket, a first elastic component, an inserting assembly, and a second elastic component. The main casing includes a position-limiting portion and a first holding portion. The bracket is disposed in the main casing and includes a second holding portion. The first elastic component is disposed between the main casing and the bracket. The first holding portion is adapted to resist an elastic force of the first elastic component to hold the bracket at a first position. The inserting assembly is disposed in the main casing. The second elastic component is disposed between the inserting assembly and the bracket. When the bracket is at the first position, the position-limiting portion limits the second holding portion, so that the second holding portion resists an elastic force of the second elastic component to hold the inserting assembly on the bracket. When the bracket is released by the first holding portion, the bracket, the second elastic component, and the inserting assembly are moved from the first position to a second position by the elastic force of the first elastic component. When the bracket is at the second position, the second holding portion is departed from the position-limiting portion, and the inserting assembly is departed from the second holding portion by the elastic force of the second elastic component and is moved to the first position.

The blood collection device of the disclosure includes a blood collection assembly and an inserting device. The inserting device includes a main casing, a bracket, a first elastic component, an inserting assembly, and a second elastic component. The main casing includes a position-limiting portion and a first holding portion. The bracket is disposed in the main casing and includes a second holding portion. The blood collection assembly is detachably disposed on the bracket. The first elastic component is disposed between the main casing and the bracket. The first holding portion is adapted to resist an elastic force of the first elastic component to hold the bracket at a first position. The inserting assembly is disposed in the main casing. The second elastic component is disposed between the inserting assembly and the bracket. When the bracket is at the first position, the position-limiting portion limits the second holding portion, so that the second holding portion resists an elastic force of the second elastic component to hold the inserting assembly on the bracket. When the bracket is released by the first holding portion, the bracket, the second elastic component, the inserting assembly, and the blood collection assembly are moved from the first position to a second position by the elastic force of the first elastic component, so that the blood collection assembly is attached to a blood collection target. When the bracket is at the second position, the second holding portion is departed from the position-limiting portion, the inserting assembly is departed from the second holding portion by the elastic force of the second elastic component and is moved to the first position, and the bracket is adapted to be departed from the blood collection assembly on the blood collection target.

In an embodiment of the disclosure, the inserting device includes a pressing component disposed on the main casing and is adapted to be pressed to drive the first holding portion to release the bracket.

In an embodiment of the disclosure, the inserting device includes a third elastic component. The third elastic component is disposed between the pressing component and the main casing, and the pressing component is adapted to resist an elastic force of the third elastic component to be pressed and is adapted to be restored by the elastic force of the third elastic component.

In an embodiment of the disclosure, the pressing component is adapted to resist the elastic force of the first holding portion to be pressed and is adapted to be restored by the elastic force of the first holding portion.

In an embodiment of the disclosure, the first holding portion includes at least one hook, and when the bracket is located at the first position, the hook is engaged with the bracket.

In an embodiment of the disclosure, the hook includes a structural reinforcement rib.

In an embodiment of the disclosure, the first holding portion includes a main casing, the main casing includes a top end and a bottom end opposite to each other, the bottom end faces the bracket, the hook extends from the top end to the bottom end by an extension length, and the extension length is less than a distance between the top end and the bottom end.

In an embodiment of the disclosure, the position-limiting portion includes a sleeve. The second holding portion includes at least one hook. When the bracket is at the first position, the hook, the inserting assembly, the second elastic component are located in the sleeve, and the hook is pushed against an inner wall of the sleeve to be engaged with the inserting assembly.

In an embodiment of the disclosure, the inserting assembly includes a main casing and a lancet. The main casing is located between the bracket and the position-limiting portion. The lancet is connected to the main casing and passes through the bracket.

In an embodiment of the disclosure, the blood collection assembly includes a sensor and a blood collection needle. The sensor is installed on the bracket. The blood collection needle is connected to the sensor. The lancet passes through the sensor and at least partially surrounds the blood collection needle.

In an embodiment of the disclosure, the blood collection assembly includes a sensor and a blood collection needle. The sensor is installed on the bracket. The blood collection needle is connected to the sensor. When the blood collection assembly is moved to the second position, the blood collection needle is adapted to enter the blood collection target, and the sensor is adapted to be attached to the blood collection target and detached from the bracket.

In summary, in the blood collection device of the embodiments, the bracket, the inserting assembly, and the blood collection assembly can be moved by the elastic force of the first elastic component and be shot at the blood collection target (e.g., the hand of a user) and after shooting, the inserting assembly is automatically restored by the elastic force of the second elastic component and back into the blood collection device to be departed from the blood collection target, so the operation of the blood collection device is facilitated. Moreover, after the bracket, the inserting assembly, and the blood collection assembly are shot at the blood collection target together to insert a blood collection needle of the blood collection assembly into the blood collection target along with the inserting assembly, the blood collection assembly can be attached to the blood collection target and departed from the bracket. Accordingly, only one operation of the blood collection device is required, the blood collection assembly is allowed to be attached to the blood collection target for a long time, and multiple blood collections can be performed repeatedly without subjecting the user to the pain caused by the inserting assembly in each blood collection. Moreover, the blood collection assembly of the embodiments is pre-installed on the bracket of the inserting assembly, so the user can use the blood collection device directly without assembling the blood collection assembly and the bracket by themselves. Therefore, the blood collection device of the embodiment has better convenience in use and can increase the willingness of users to collect blood by themselves.

### BRIEF DESCRIPTION OF THE DRAWINGS

FIG. 1 is a perspective view of a blood collection device according to an embodiment of the disclosure.

FIG. 2 is an exploded view of the blood collection device of FIG. 1.

FIG. 3 is a perspective view of part of the structure of the blood collection device of FIG. 1.

FIG. 4A and FIG. 4B illustrate the operation of the blood collection device of FIG. 3.

FIG. 5 is a perspective view of part of the structure of a blood collection device according to another embodiment of the disclosure.

FIG. 6 is a perspective view of part of the structure of a blood collection device according to another embodiment of the disclosure.

### DESCRIPTION OF THE EMBODIMENTS

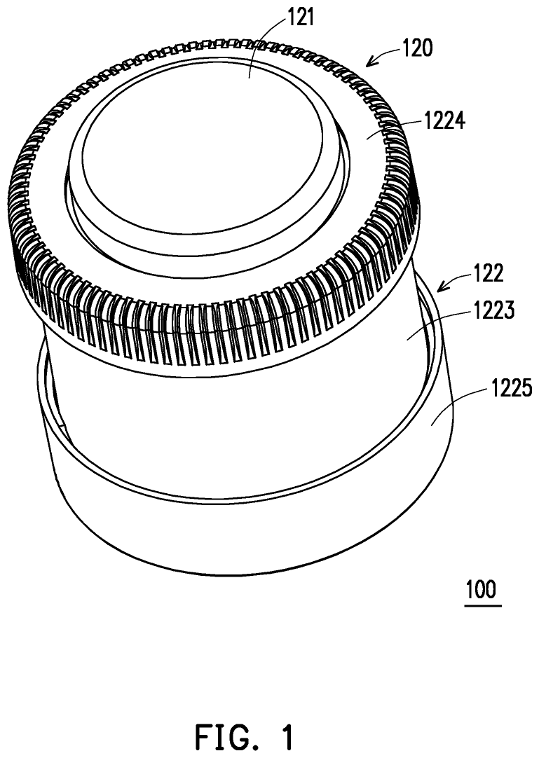

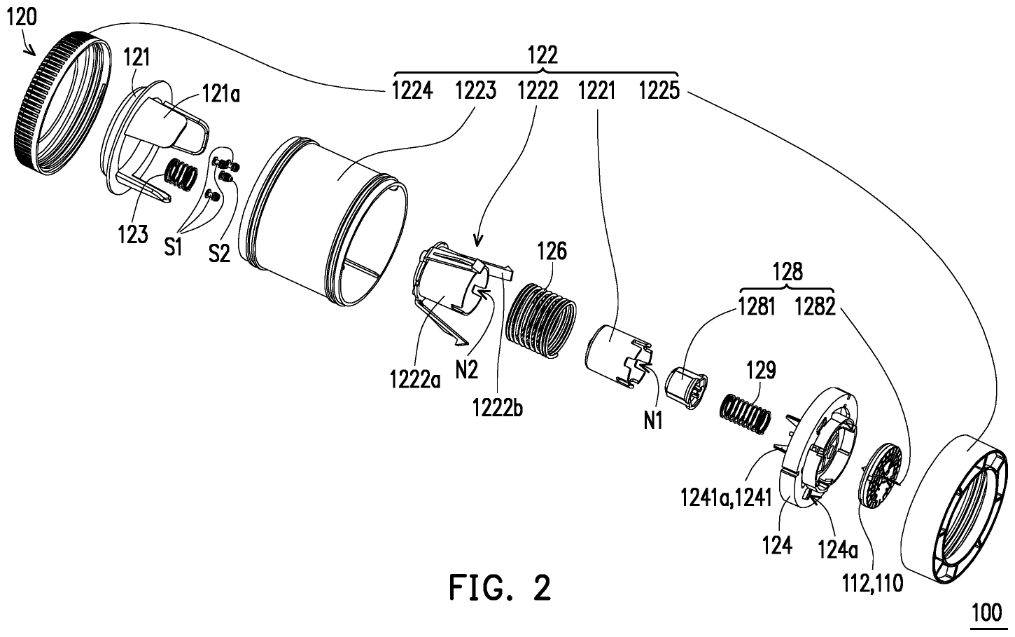

FIG. 1 is a perspective view of a blood collection device according to an embodiment of the disclosure. FIG. 2 is an exploded view of the blood collection device of FIG. 1. Referring to FIG. 1 and FIG. 2, a blood collection device 100 of the embodiment includes a blood collection assembly 110 and an inserting device 120. The inserting device 120 includes a main casing 122, a bracket 124, a first elastic component 126, an inserting assembly 128, and a second elastic component 129. The main casing 122 has a position-limiting portion 1221 and a first holding portion 1222.

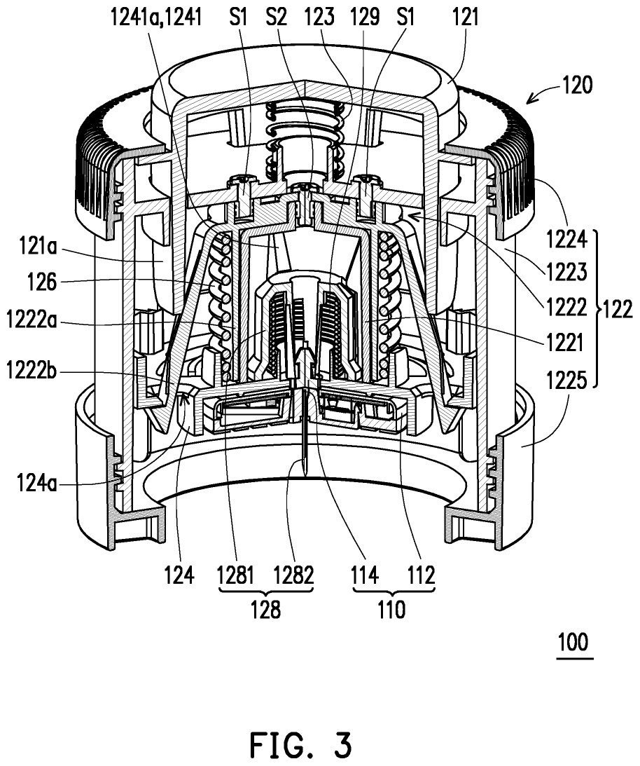

FIG. 3 is a perspective view of part of the structure of the blood collection device of FIG. 1. Referring to FIG. 2 and FIG. 3, the bracket 124 is disposed in the main casing 122 and has a second holding portion 1241. The blood collection assembly 110 is detachably disposed on the bracket 124. The first elastic component 126 is disposed between the main casing 122 and the bracket 124, and the first holding portion 1222 is adapted to resist the elastic force of the first elastic component 126 to hold the bracket 124 at the first position shown in FIG. 3. The inserting assembly 128 is disposed in the main casing 122, and the second elastic component 129 is disposed between the inserting assembly 128 and the bracket 124. When the bracket 124 is at the first position shown in FIG. 1, the position-limiting portion 1221 limits the second holding portion 1241, such that the second holding portion 1241 resists the elastic force of the second elastic component 129 to hold the inserting assembly 128 on the bracket 124.

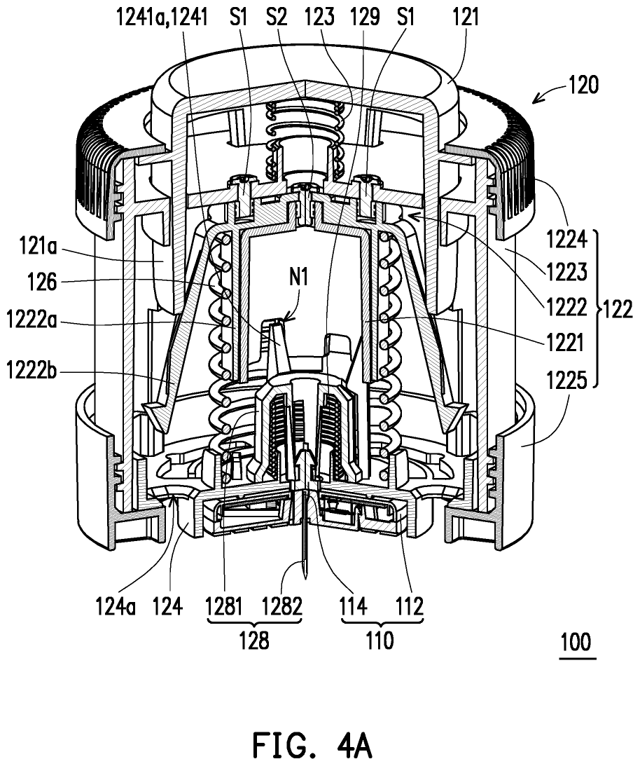

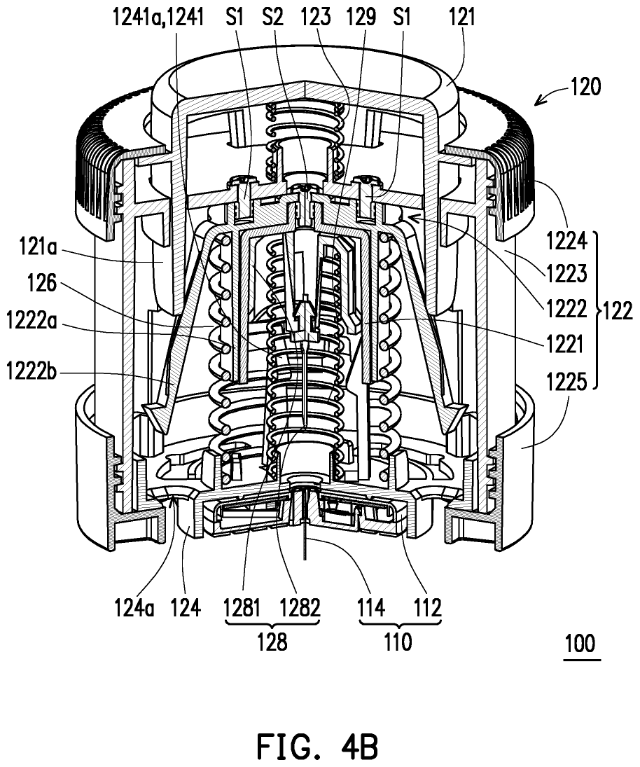

FIG. 4A and FIG. 4B illustrate the operation of the blood collection device of FIG. 3. When the bracket 124 is released by the first holding portion 1222, the bracket 124, the inserting assembly 128, the second elastic component 129, and the blood collection assembly 110 are moved from the first position shown in FIG. 3 to the second position shown in FIG. 4A by the elastic force of the first elastic component 126, such that the blood collection assembly 110 is attached to a blood collection target (e.g., a user's hand) at the bottom end of the blood collection device 100. When the bracket 124 is at the second position shown in FIG. 4A, the second holding portion 1241 is departed from the position-limiting portion 1221, the inserting assembly 128 is departed from the second holding portion 1241 by the elastic force of the second elastic component 129 and is moved toward the first position as shown in FIG. 4B, and the bracket 124 is adapted for being departed from the blood collection assembly 110 on the blood collection target.

As described above, in the blood collection device 100 of the embodiment, the bracket 124, the inserting assembly 128, the second elastic component 129, and the blood collection assembly 110 can be moved by the elastic force of the first elastic component 126 to shoot at the blood collection target at the bottom end of the blood collection device 100, and after shooting, the inserting assembly 128 is automatically restored by the elastic force of the second elastic component 129 and back into the blood collection device 100 to be departed from the blood collection target, so the operation of the blood collection device 100 is facilitated. Moreover, after the bracket 124, the inserting assembly 128, and the blood collection assembly 110 are shot at the blood collection target together to insert a blood collection needle 114 of the blood collection assembly 110 into the blood collection target along with the inserting assembly 128, the blood collection assembly 110 can be attached to the blood collection target and departed from the bracket 124. Accordingly, only one operation of the blood collection device 100 is required, the blood collection assembly 110 is allowed to be attached to the blood collection target for a long time, and multiple blood collections can be performed repeatedly without subjecting the user to the pain caused by the inserting assembly 128 in each blood collection. Moreover, the blood collection assembly 110 of the embodiment is pre-installed on the bracket 124 of the inserting assembly 120, so the user can use the blood collection device 100 directly without assembling the blood collection assembly 110 and the bracket 124 by themselves. Therefore, the blood collection device 100 of the embodiment has better convenience in use and can increase the willingness of users to collect blood by themselves.

The composition and operation mode of each component of the blood collection device 100 of the embodiment may be illustrated in detail in the subsequent paragraphs.

In the embodiment, the inserting device 120 further includes a pressing component 121 and a third elastic component 123. The pressing component 121 is disposed on the main casing 122, and the third elastic component 123 is disposed between the pressing component 121 and the main casing 122. The pressing component 121 is adapted to resist the elastic force of the third elastic component 123 and to drive the first holding portion 1222 to release the bracket 124 when being pressed by the user, and the pressing component 121 is adapted to be restored by the elastic force of the third elastic component 123. In other embodiments, other structures and methods may be used to drive the first holding portion 1222 to release the bracket 124, and the disclosure is not limited thereto.

More specifically, the main casing 122 of the embodiment includes a housing 1223, an upper cover 1224, and a lower cover 1225. The housing 1223 accommodates the position-limiting portion 1221, the first holding portion 1222, the first elastic component 126, the inserting assembly 128, the second elastic component 129, and the blood collection assembly 110. For example, the upper cover 1224 is a screw cover, which is fixed to the top of the housing 1223 in a rotary screwing manner, and is used to stop the pressing component 121 to prevent the pressing component 121 from being departed from the main casing 122. For example, the lower cover 1225 is a screw cover, which is fixed to the bottom of the housing 1223 in a rotary screwing manner, and is used to stop the bracket 124 to prevent the bracket 124 from being departed from the main casing 122.

The first holding portion 1222 is fixed to the housing 1223 by a screw S1, and the position-limiting portion 1221 is fixed to the housing 1223 and the first holding portion 1222 by a screw S2. The first holding portion 1222 includes a main casing 1222a and at least one hook 1222b (multiple hooks are shown), and the hook 1222b is connected to the main casing 1222a. The main casing 1222a is used for accommodating the position-limiting portion 1221. The hook 1222b is used to be engaged with a slot 124a of the bracket 124 to hold the bracket 124 as shown in FIG. 3. The user needs to resist the elastic force of the third elastic component 123 and the elastic force of the hook 1222b of the first holding portion 1222 to press the pressing component 121, the pressed pressing component 121 exerts force on the hook 1222b by its extension 121a, so that the hook 1222b is elastically deformed toward the main casing 1222a to release the bracket 124. When the user no longer presses the pressing component 121, the pressing component 121 is not only restored upward by the elastic force of the third elastic component 123 but also by the elastic force of the hook 1222b of the first holding portion 1222. In other embodiments, the configuration of the third elastic component 123 may be omitted, and the pressing component 121 can be restored upward only by the elastic force of the hook 1222b.

The second holding portion 1241 of the bracket 124 includes at least one hook 1241a (multiple hooks are shown). For example, the position-limiting portion 1221 is a sleeve. When the bracket 124 is at the first position shown in FIG. 3, the hook 1241a, the inserting assembly 128, and the second elastic component 129 are located in the sleeve (position-limiting portion 1221), and the hook 1241a is pushed against the inner wall of the sleeve (position-limiting portion 1221) to be engaged with the inserting assembly 128. Moreover, the sleeve (position-limiting portion 1221) has at least one notch N1 (multiple notches are shown in FIG. 2), and the main casing 1222a of the first holding portion 1222 has at least one notch N2 (multiple notches are shown in FIG. 2). When the bracket 124 is at the second position as shown in FIG. 4A, the hook 1241a is released by the sleeve (position-limiting portion 1221) corresponding to the notch N1 and the notch N2, and the inserting assembly 128 of FIG. 4A can push the hook 1241a outward and move upward to the position shown in FIG. 4B by the elastic force of the second elastic component 129.

The inserting assembly 128 includes a main casing 1281 and a lancet 1282. The main casing 1281 is located between the bracket 124 and the position-limiting portion 1221, and the lancet 1282 is connected to the main casing 1281. The blood collection assembly 110 includes a sensor 112 and the blood collection needle 114. The sensor 112 is detachably disposed on the bracket 124 by a buckle or other suitable methods, and the blood collection needle 114 is connected to the sensor 112. The lancet 1282 of the inserting assembly 128 passes through the bracket 124 and the sensor 112 and at least partially surrounds the blood collection needle 114.

The hardness of the lancet 1282 is greater than the hardness of the blood collection needle 114. Therefore, when the bracket 124, the inserting assembly 128, and the blood collection assembly 110 are shot together to reach the second position as shown in FIG. 4A, the lancet 1282 can be inserted into the blood collection target (e.g., the hand of a user), the blood collection needle 114 then enters the blood collection target, and the sensor 112 is attached to the surface of the blood collection target by the glue at the bottom thereof. Then, when the inserting assembly 128 is restored upward as shown in FIG. 4B by the elastic force of the second elastic component 129, the lancet 1282 is departed from the blood collection target and departed from the blood collection needle 114. The bonding force between the glue at the bottom of the sensor 112 and the blood collection target is greater than the bonding force between the bracket 124 and the sensor 112, for example. Therefore, when the sensor 112 shown in FIG. 4B is attached to the surface of the blood collection target, the user may only need to apply an upward force to the inserting device 120 to separate the bracket 124 and the sensor 112 from each other, so that the blood collection assembly 110 stays on the blood collection target for subsequently collecting blood repeatedly for a long time.

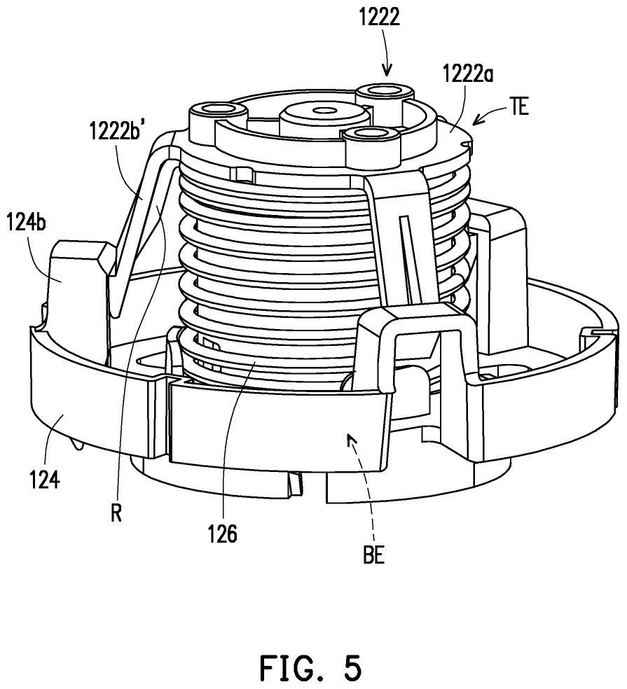

FIG. 5 is a perspective view of part of the structure of a blood collection device according to another embodiment of the disclosure. The difference between the embodiment shown in FIG. 5 and the previous embodiment is that the bracket 124 of FIG. 5 has multiple engaging portions 124b extending upward, and hooks 1222b′ of the first holding portion 1222 are engaged to the engaging portions 124b. With this configuration, the hook 1222b′ can have a shorter length. Specifically, the main casing 1222a of the first holding portion 1222 has a top end TE and a bottom end BE opposite to each other. The bottom end BE faces the bracket 124, the hook 1222b′ extends from the top end TE to the bottom end BE by an extension length, and this extension length is less than the distance between the top end TE and the bottom end BE. Moreover, the hook 1222b′ may have a structural reinforcement rib R to improve the structural strength of the hook 1222b′.

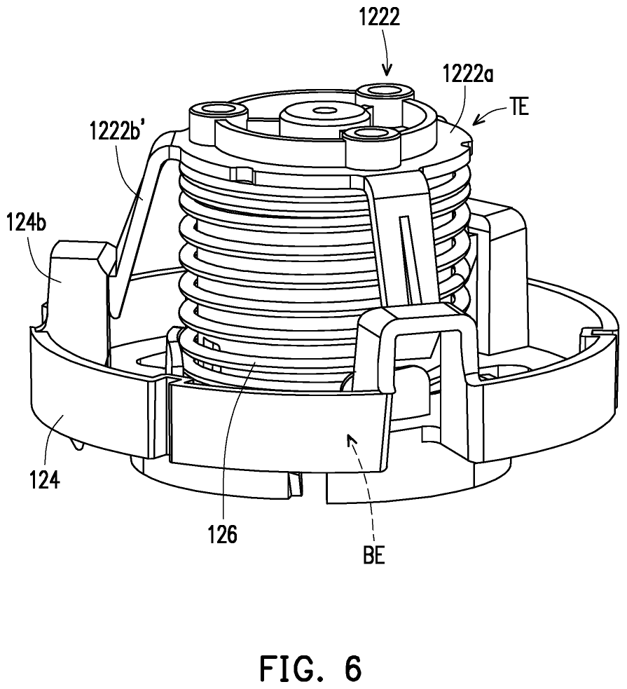

FIG. 6 is a perspective view of part of the structure of a blood collection device according to another embodiment of the disclosure. The difference between the embodiment shown in FIG. 6 and the embodiment shown in FIG. 5 is that the hook 1222b′ of FIG. 6 does not have the structural reinforcing rib R.

In summary, in the blood collection device of the embodiments, the bracket, the inserting assembly, the second elastic component, and the blood collection assembly can be moved by the elastic force of the first elastic component and be shot at the blood collection target (e.g., the hand of a user) and after shooting, the inserting assembly is automatically restored by the elastic force of the second elastic component and back into the blood collection device to be departed from the blood collection target, so the operation of the blood collection device is facilitated. Moreover, after the bracket, the inserting assembly, and the blood collection assembly are shot at the blood collection target together to insert a blood collection needle of the blood collection assembly into the blood collection target along with the inserting assembly, the blood collection assembly can be attached to the blood collection target and departed from the bracket. Accordingly, only one operation of the blood collection device is required, the blood collection assembly is allowed to be attached to the blood collection target for a long time, and multiple blood collections can be performed repeatedly without subjecting the user to the pain caused by the inserting assembly in each blood collection. Moreover, the blood collection assembly of the embodiments is pre-installed on the bracket of the inserting assembly, so the user can use the blood collection device directly without assembling the blood collection assembly and the bracket by themselves. Therefore, the blood collection device of the embodiments has better convenience in use and can increase the willingness of users to collect blood by themselves.

What is claimed is:

1. An inserting device, comprising:
a main casing with a position-limiting portion and a first holding portion;
a bracket disposed in the main casing and comprising a second holding portion;
a first elastic component disposed between the main casing and the bracket, wherein the first holding portion is adapted to resist an elastic force of the first elastic component to hold the bracket at a first position;
an inserting assembly disposed in the main casing; and
a second elastic component disposed between the inserting assembly and the bracket, wherein when the bracket is at the first position, the position-limiting portion limits the second holding portion, so that the second holding portion resists an elastic force of the second elastic component to hold the inserting assembly on the bracket,
when the bracket is released by the first holding portion, the bracket, the second elastic component, and the inserting assembly are moved from the first position to a second position by the elastic force of the first elastic component,
when the bracket is at the second position, the second holding portion is departed from the position-limiting portion, and the inserting assembly is departed from the second holding portion by the elastic force of the second elastic component and is moved to the first position.

2. The inserting device according to claim 1, comprising a pressing component, wherein the pressing component is disposed on the main casing and is adapted to be pressed to drive the first holding portion to release the bracket.

3. The inserting device according to claim 2, comprising a third elastic component, wherein the third elastic component is disposed between the pressing component and the main casing, and the pressing component is adapted to resist an elastic force of the third elastic component to be pressed, and is adapted to be restored by the elastic force of the third elastic component.

4. The inserting device according to claim 2, wherein the pressing component is adapted to resist the elastic force of the first holding portion to be pressed, and is adapted to be restored by the elastic force of the first holding portion.

5. The inserting device according to claim 1, wherein the first holding portion comprises at least one hook, and when the bracket is located at the first position, the at least one hook is engaged with the bracket.

6. The inserting device according to claim 5, wherein the at least one hook comprises a structural reinforcement rib.

7. The inserting device according to claim 5, wherein the first holding portion comprises a main casing, the main casing comprises a top end and a bottom end opposite to each other, the bottom end faces the bracket, the at least one hook extends from the top end to the bottom end by an extension length, and the extension length is less than a distance between the top end and the bottom end.

8. The inserting device according to claim 1, wherein the position-limiting portion comprises a sleeve; the second holding portion comprises at least one hook; when the bracket is at the first position, the at least one hook, the inserting assembly, the second elastic component are located in the sleeve; and the at least one hook is pushed against an inner wall of the sleeve to be engaged with the inserting assembly.

9. The inserting device according to claim 1, wherein the inserting assembly comprises a main casing and a lancet, the main casing is located between the bracket and the position-limiting portion, and the lancet is connected to the main casing and passes through the bracket.

10. A blood collection device, comprising:
a blood collection assembly; and
an inserting device, comprising:
a main casing with a position-limiting portion and a first holding portion;
a bracket disposed in the main casing and comprising a second holding portion, wherein the blood collection assembly is detachably disposed on the bracket;
a first elastic component disposed between the main casing and the bracket, wherein the first holding portion is adapted to resist an elastic force of the first elastic component to hold the bracket at a first position;
an inserting assembly disposed in the main casing; and
a second elastic component disposed between the inserting assembly and the bracket, wherein when the bracket is at the first position, the position-limiting portion limits the second holding portion, so that the second holding portion resists an elastic force of the second elastic component to hold the inserting assembly on the bracket,
when the bracket is released by the first holding portion, the bracket, the second elastic component, the inserting assembly, and the blood collection assembly are moved from the first position to a second position by the elastic force of the first elastic component, so that the blood collection assembly is attached to a blood collection target,
when the bracket is at the second position, the second holding portion is departed from the position-limiting portion, the inserting assembly is departed from the second holding portion by the elastic force of the second elastic component and is moved to the first position, and the bracket is adapted to be departed from the blood collection assembly on the blood collection target.

11. The blood collection device according to claim 10, wherein the inserting device comprises a pressing component disposed on the main casing and adapted to be pressed to drive the first holding portion to release the bracket.

12. The blood collection device according to claim 11, wherein the inserting device comprises a third elastic component, the third elastic component is disposed between the pressing component and the main casing, and the pressing component is adapted to resist an elastic force of the third elastic component to be pressed and is adapted to be restored by the elastic force of the third elastic component.

13. The blood collection device according to claim 11, wherein the pressing component is adapted to resist the elastic force of the first holding portion to be pressed, and is adapted to be restored by the elastic force of the first holding portion.

14. The blood collection device according to claim 10, wherein the first holding portion comprises at least one hook, and when the bracket is located at the first position, the at least one hook is engaged with the bracket.

15. The blood collection device according to claim 14, wherein the at least one hook comprises a structural reinforcement rib.

16. The blood collection device according to claim 14, wherein the first holding portion comprises a main casing, the main casing comprises a top end and a bottom end opposite to each other, the bottom end faces the bracket, the at least one hook extends from the top end to the bottom end by an extension length, and the extension length is less than a distance between the top end and the bottom end.

17. The blood collection device according to claim 10, wherein the position-limiting portion comprises a sleeve; the second holding portion comprises at least one hook; when the bracket is at the first position, the at least one hook, the inserting assembly, the second elastic component are located in the sleeve; and the at least one hook is pushed against an inner wall of the sleeve to be engaged with the inserting assembly.

18. The blood collection device according to claim 10, wherein the inserting assembly comprises a main casing and a lancet, the main casing is located between the bracket and the position-limiting portion, and the lancet is connected to the main casing and passes through the bracket

19. The blood collection device according to claim 18, wherein the blood collection assembly comprises a sensor and a blood collection needle, the sensor is installed on the bracket, the blood collection needle is connected to the sensor, and the lancet passes through the sensor and at least partially surrounds the blood collection needle.

20. The blood collection device according to claim 18, wherein the blood collection assembly comprises a sensor and a blood collection needle, the sensor is installed on the bracket, the blood collection needle is connected to the sensor, when the blood collection assembly is moved to the second position, the blood collection needle is adapted to enter the blood collection target, and the sensor is adapted to be attached to the blood collection target and detached from the bracket.
